(19) United States
(12) Patent Application Publication
(10) Pub. No.: US 2003/0166549 A1
(43) Pub. Date: Sep. 4, 2003

(54) Targeting peptides

(76) Inventors: Klaus Schughart, Kenzingen (DE); Ulla Rasmussen, Achenheim (FR); Valerie Schreiber, Graffestaden (FR)

Correspondence Address: BURNS DOANE SWECKER & MATHIS L L P, POST OFFICE BOX 1404, ALEXANDRIA, VA, 22313-1404, US

(21) Appl. No.: 10/182,573

(22) PCT Filed: Jan. 26, 2001
(86) PCT No.: PCT/EP01/00894

(30) Foreign Application Priority Data
Feb. 2, 2000 (EP) 00440030.5
Aug. 21, 2000 (EP) 00440229.3

(51) Int. Cl. A61K 38/17; A61K 38/10; A61K 38/08; C07K 14/00; C07K 7/08

(52) U.S. Cl. 514/12; 514/13; 514/14; 514/15; 530/324; 530/325; 530/326; 530/327

(57) ABSTRACT

The present invention relates to targeting peptides and more specifically peptides that target heart and various tumors as well as to their use for targeting. The present invention also provides a composition comprising at least one therapeutic agent and at least one peptide of the invention or, alternatively, a nucleic acid molecule encoding such a peptide as well as its use for the preparation of a drug intended for gene transfer.

The present invention provides novel peptides and, more particularly peptides that are able to target preferentially heart and various tumor cells. The present invention also relates to a composition comprising such a peptide and a therapeutic agent. The invention is of very special interest in relation to prospect for gene therapy, in particular in human.

Gene therapy can be defined as the transfer of genetic material into a cell or an organism to treat or prevent a cell deficiency or insufficiency. The possibility of treating human disorders by gene therapy has changed in a few years from the stage of theoretical considerations to that of clinical applications. The first protocol applied to man was initiated in the USA in September 1990 on a patient who was genetically immunodeficient as a result of a mutation affecting the gene encoding adenine deaminase (ADA) and the relative success of this first experiment encouraged the development of the technology for various inherited as well as acquired diseases.

Successful gene therapy depends on the efficient delivery of a therapeutic gene to the cells of a living organism and expression of the genetic information. Functional genes can be introduced into cells by a variety of techniques resulting in either transient expression (transient transfection) or permanent transformation of the host cells with incorporation into the host genome. Whereas direct injection of naked nucleic acids (i.e. plasmidic DNA) can be envisaged (Wolff et al., Science 247 (1990) 1465-1468), the majority of the protocols uses vectors to carry the genes of interest. The vectors can be divided into two categories.

The first category relates to viral vectors, especially adeno- and retroviral vectors. Viruses have developed diverse and highly sophisticated mechanisms to achieve transport across the cellular membrane, escape from lysosomal degradation, delivery of their genome to the nucleus and, consequently, have been used in many gene delivery applications. Their structure, organization and biology are described in the literature available to a person skilled in the art.

One of the most widely used vectors for in vivo gene transfer is a replication-deficient recombinant adenoviral vector. Some of its advantages are the fact that it can be grown to high titers and can efficiently transduce a wide variety of human cell types. The adenoviral genome consists of a linear double-standed DNA molecule of approximately 36 kb carrying more than about thirty genes necessary to complete the viral cycle. The early genes are divided into 4 regions (E1 to E4) that are essential for viral replication with the exception of the E3 region, which is believed to modulate the anti-viral host immune response. The late genes encode in their majority the structural proteins constituting the viral capsid. In addition, the adenoviral genome carries at both extremities cis-acting 5′ and 3′ ITR (Inverted Terminal Repeat) and packaging sequences essential for DNA replication. The adenoviral vectors used in gene therapy protocols lack most of the E1 region in order to avoid their replication and subsequent dissemination in the environment and the host body. Additional deletions in the E3 region increase the cloning capacity (for a review see for example Yeh et al. FASEB Journal 11 (1997) 615-623). Second generation vectors retaining the ITRs and packaging sequences and containing substantial genetic modifications aimed to abolish the residual synthesis of the viral antigens, are currently constructed in order to improve long-term expression of the therapeutic gene in the transduced cells (WO94/28152, Lusky et al. J. Virol 72 (1998) 2022-2032).

The specificity of infection of the adenoviruses is determined by the attachment of the virions to cellular receptors present at the surface of the permissive cells. In this regard, the fiber present at the surface of the viral capsid play a critical role in cellular attachment (Defer et al. J. Virol. 64 (1990) 3661-3673) and penton-base promotes internalization through the binding to the cellular integrins (Mathias et al. J. Virol. 68 (1994) 6811-6814). Recent studies have presumed the use of the coxsackie virus receptor (CAR) by the types 2 and 5 adenoviruses (Bergelson et al; Science 275 (1997) 1320-1323). However, other surface proteins may be involved in fiber attachment, for example, the α2 domain of the class I histocompatibility antigens as identified by Hong et al. (EMBO J. 16 (1997) 2294-2306). The fiber is composed of 3 regions (Chroboczek et al. Current Top. Microbiol. Immunol. 199 (1995) 165-200 ): the tail at the N-terminus of the protein which interacts with penton base and ensures the anchorage in the capsid, the shaft composed of a number of β-sheets repeats and the knob which contains the trimerization signals (Hong et al. J. Virol. 70 (1996) 7071-7078) and the receptor binding moiety (Henri et al. J. Virol. 68 (1994) 5239-5246; Louis et al. J. Virol. 68 (1994) 4104-4106).

The second category relates to synthetic vectors. A large number derived from various lipids and polymers are currently available (for a review, see for example Rolland, Critical reviews in Therapeutic Drug Carrier Systems 15 (1998) 143-198). Although less efficient than viral delivery systems, they present potential advantages with respect to large-scale production, safety, low immunogenicity and cloning capacity. Moreover, they can be easily modified by simple mixing of the desired components.

The design of viral and synthetic gene therapy vectors which are capable to deliver therapeutic genes to a specific cell represents one of the main interest and challenge in today's gene therapy research. The use of targeting vectors would limit the vector spread, thus increasing therapeutic efficacy for the desired target cells and minimizing potential side effects. Targeting can be achieved by first identifying a suitable address at the cellular surface and then modifying the vectors in such a way that they can recognize this address.

It has been shown that a cell type or a disease affected cell expresses unique cell surface markers. For example, endothelial cells in rapidely growing tumors express cell surface proteins not present in quiescent endothelium, i.e. αv integrins (Brooks et al. Science 264 (1994) 569) and receptors for certain angiogenic growth factors (Hanahan Science 277 (1997) 48). Phage display library selection methods can be employed to select peptide sequences that interact with these particular cell surface markers (see for example U.S. Pat. No. 5,622,699 U.S. Pat. No. 5,223,409 and U.S. Pat. No. 5,403,484). In this system, a random peptide is expressed on the phage surface by fusion of the corresponding coding sequence to a gene encoding one of the phage surface proteins. The desired phages are selected on the basis of their binding to the target such as isolated organ fragments (ex vivo procedure), as for example exemplified in WO 98/39469 and WO 99/45020, or cultured cells (in vitro procedure). Identification of targeting peptides can also be done by an in vivo procedure that is achieved by injecting phage libraries into mice and subsequently rescuing the bound phages from the targeted organs. Selected peptides are identified by sequencing the genome phage region encoding the displayed peptide. In vivo organ screening was successfully applied to isolate peptide sequences that conferred selective phage homing to the brain and kidney (Pasqualini et al., Nature 380 (1996) 364-366; WO 97/10507), to the vasculature of lung, skin and pancreas (Rajotte et al., J. Clin. Invest. 102 (1998) 430-437) and to several tumor types (Pasqualini et al., Nature Biotechnology 15 (1997) 542-546).

Furthermore, tumors could be targeted not only via their vasculature but also via the extracellular matrix (ECM) or the tumor cells themselves. Since blood vessels are constantly modified in tumors, the endothelium is locally disrupted allowing gene therapy vectors to extravasate and interact with the ECM and tumor cells. Peptides which interact with the ECM or tumor-associated cell surface markers could also be selected using the phage display technique (Christiano et al. Cancer Gene Therapy 3 (1996) 4-10; Croce et al. Anticancer Res. 17 (1997) 4287-4292; Gottschalk et al. Gene Ther. 1 (1994) 185-191; Park et al. Adv Pharmacol. 40 (1997) 399-435). As an example, a HWGF motif was identified as a ligand of the matrix metalloproteinases involved in tumor growth, angiogenesis and metastasis. Administration of a HWGF comprising peptide to a tumour bearing animal model prevents tumor growth and invasion and prolongs animal survival (Koivunen et al. Nature Biotechnology 17 (1999) 768-774).

Recently, Romanczuk et al. (Human Gene Therapy 10 (1999) 2615-2626) reported the isolation of peptides targeting the differentiated, cilliated airway epithelial cells. Coupling of the best binding peptide to the surface of a recombinant adenovirus with bifunctional polyethylene glycol (PEG) resulted in a vector able to transduce the target cells via an alternative pathway dependent on the incorporated peptide.

All together, very few cell type or disease-specific surface markers have been described up to now and only very few ligands are known that specifically interact with such markers. Meanwhile, a molecule that selectively homes to lung endothelium, in particular to membrane dipeptidase, has been identified (see for example WO 99/46284). Therefore, the technical problem underlying the present invention is the provision of improved methods and means for the targeting of therapeutic agents to specific cells.

This technical problem is solved by the provision of the embodiments as defined in the claims.

Accordingly, the present invention relates to a peptide selected from the group consisting of:

X₁THPRFATX₂ (SEQ ID NO:1)

X₁RTPFATYX₂ (SEQ ID NO:2)

X₁FHVNPTSPTHPLX₂ (SEQ ID NO:3)

X₁QTSSPTPLSHTQX₂

X₁PQTSTLLX₂

X₁HLPTSSLFDTTHX₂

X₁VHHLPRTX₂

X₁QLHNHLPX₂

X₁HSFDHLPAAALHX₂

X₁YPSAPPQWLTNTX₂

X₁YPSQSQRX₃LSX₄HX₂

X₁TYPSSTLX₂

X₁NTLQVRGVYPSVX₂

X₁YSNRTNTNSHWAX₂

X₁PATNTSKX₂

X₁HVNKLHGX₂

X₁FHVNPTSPTHPLX₂

X₁NANKLWTWVSSPX₂

X₁SGRIPYLX₂

X₁NEDINDVSGRLSX₂

X₁LSPQRASQRLYSX₂

X₁SFSTSPQX₂

X₁ERMDSPQX₂

X₁HHGHSPTSPQVRX₂

X₁GSSTGPQRLHVPX₂

X₁TCSLCNPVQPQRX₂

X₁QRLTTLYX₂

X₁WSPGQQRLHNSTX₂

X₁WKSELPVQRARFX₂

X₁SELPSMRLYTQPX₂

X₁HSLHVHKGLSELX₂

X₁SDLPVQLEPERQX₂

X₁TRYLPVLPSLFPX₂

X₁TCSLCNPVQPQRX₂

X₁WEPPVQSAWQLSX₂

X₁HFTFPQQQPPRPX₂

X₁GSTSRPQPPSTVX₂

X₁NFSQPPSKHTRSX₂

X₁QYPHKYTLQPPKX₂

X₁FNQPPSWRVSNSX₂

X₁SVSVGMKPSPRPX₂

X₁STPRPPLGIPAQX₂

X₁TQSPLNYRPALLX₂

X₁AQSPTIKLTPSWX₂

X₁HNLLTQSX₂

X₁TLVQSPMX₂

X₁NLNTDNYRQLRHX₂

X₁FRPAVHNMPSLQX₂

X₁ISRPAPISVDMKX₂

X₁THRPSLPDSSRAX₂

X₁ALHPLTHRHYATX₂

X₁THRGPQSX₂

X₁SFHMPSRAVSLSX₂

X₁NQSNFTSRALLYX₂

X₁SFPTHIDHHVSPX₂

X₁LNGDPTHX₂

X₁HMPHHVSNLQLHX₂

X₁LPSVSPVLQVLGX₂

X₁DAQQLYLSNWRSX₂

X₁DSYLSSTLPGQLX₂

X₁SPTPTSHQQLHSX₂

X₁APPGNWRNYLMPX₂

X₁LSNKMSQX₂

X₁MHNVSDSNDSAIX₂

X₁DNSNDLMX₂

X₁TVMEAPRSAILIX₂

X₁CNDIGWVRCX₂

X₁CWPYPSHFCX₂

X₁MPLPQPSHLPLLX₂

X₁LPQRAFWVPPIVX₂

X₁WPVRPWMPGPVVX₂

X₁WPTSPWLEREPAX₂

X₁WPTSPWSSRDWSX₂

X₁HEWSYLAPYPWFX₂

X₁QIDRWFDAVQWLX₂

X₁CLPSTRWTCX₂

X₁CWPMKSX₅FCX2

wherein each X₁ and X₂ independently of one another represents any amino acid sequence of n amino acids, n varying from 0 to 50 and n being identical or different in X₁ and X₂, and wherein X₃, X₄ and X₅, identical or different, represent any amino acid. Preferably, X₅ is a leucine (L) or a glutamine (Q) residue.

These peptides are useful to direct e.g., gene therapy vectors to specific targets in an organism.

The terms <<peptide>> and <<amino acid>> used herein are intended to have the same meaning as commonly understood by one of ordinary skill in the art. According to a preferred embodiment, n is ranging independently of one another in X₁ and X₂ from 0 to about 10 amino acids and more preferably from 0 to about 5 amino acids. Peptides according to the invention may be produced de novo by synthetic methods or by expression of the appropriate DNA fragment by recombinant DNA techniques in eukaryotic as well as prokaryotic cells. Alternatively, they can also be produced by fusion to a fusion partner. When the fusion partner is a polypeptide, fusion can be designed to place the peptide at the N- or C terminus or between two residues of said polypeptide.

The peptide according to the invention can be purified by art known techniques such as reverse phase chromatography, size exclusion, high performance liquid chromatography, ion exchange chromatography, gel electrophoresis, affinity chromatography and the like. The conditions and technology used to purify a particular peptide of the invention will depend on the synthesis method and on factors such as net charge, hydrophobicity, hydrophilicity and will be apparent to those having skill in the art.

Optionally, the peptide of the invention may include modifications of one or more amino acid residue(s) by way of substitution or addition of moieties (i.e. glycosylation, alkylation, acetylation, amidation, phosphorylation and the like). Included within the scope of the present invention are for example peptides containing one or more analogs of an amino acid (including not naturally occuring amino acids), peptides with substituted linkages as well as other modifications known in the art both naturally occurring and non naturally occuring. The peptide can be linear or cyclized for example by flanking the peptide at both extremities by cysteine residues. In accordance with the aim pursued with the present invention, preferred modifications are those that allow or improve the coupling of a peptide of the invention to a therapeutic agent as described hereinafter (i.e. addition of sulfhydryl, amine groups . . . ). The present invention also encompasses analogs of a peptide according to the invention where at least one amino acid is replaced by another amino acid having similar properties. The matrix of FIGS. 84 and 85 of Atlas of Protein Sequence and Structure (1978, Vol. 5, ed. M. O. Dayhoff, National Biomedical Research Foundation, Washington, D.C.) show the groups of chemically similar amino acids that tend to replace one another: the hydrophobic group; the aromatic group; the basic group; the acid, acid-amide group; cysteine; and the other hydrophilic residues. Analogs can also be retro or inverso peptides (WO95/24916).

The present invention also contemplates modifications that render the peptides of the invention detectable. For this purpose, the peptides of the invention can be modified with a detectable moiety (i.e. a scintigraphic, radioactive, fluorescent, or dye labels and the like). Suitable radioactive labels include but are not limited to Tc^{99m}, I¹²³ and In¹¹¹. Such labels can be attached to the peptide of the invention in known manner, for example via a cysteine residue. Other techniques are described elsewhere.

The peptides of the invention may be used for a variety of purposes.

According to a first and preferred alternative (second embodiment), a peptide of the invention may be used for targeting purposes. Targeting is defined as the capability of recognizing and binding preferentially to a cell intended to be targeted. <<Preferentially>> means that the peptide of the invention provides lesser attachment to a non target cell compared to a target cell. Generally, a particular peptide of the invention recognizes and binds a marker that is expressed or exposed at the surface of such a cell (i.e. cell surface marker, receptor, peptide presented by the histocompatibility antigens, tumor-specific antigen . . . ). Within the framework of the present invention, it may be advantageous to target more particularly a tumor cell, a particular cell type or a category of cells. Examples of particular cell types include but are not limited to liver and heart cells. Categories of cells include cells of artherosclerotic plaques, ischaemic regions, parenchyme, ECM, vasculature, coronary artery.

A second alternative is a use related to the study, isolation and purification of the cell surface markers to which such peptides specifically bind. Another alternative relates to diagnostic purposes for example for imaging the target cells exhibiting such markers by in vitro as well as in vivo assays. Accordingly, the scope of the present invention also includes a diagnostic reagent for detection of a target cell, said reagent comprising a peptide according to the invention and a carrier. Preferably, the peptide is modified with a detectable moiety and the carrier is for systemic injection.

Finally, a peptide according to the invention may be used for therapeutic as well as prophylactic purposes, intended for the treatment of the human or animal body. A peptide according to the present invention may have therapeutic effects by itself (i.e. angiostatic, inhibitors of metalloproteases, cell-cycle inhibitors, cytostatic, cytotoxic, endosome reduction, membranolytic, proliferation-inducing properties . . . ) in addition to its targeting properties (see for example Koivunen et al. Nat. Biotech. 17 (1999) 768-774).

According to a third embodiment, the present invention also provides peptides for heart targeting. A heart targeting peptide of the invention has a minimal size of 7 amino acids. Such peptides can be classified in different families that are defined according to the presence of some common amino acid motifs. Each peptide in a family contains a particular motif but in a different amino acid environment. The present invention also encompasses the case where a particular peptide comprises more than one selected motif that can be continous, separated by a stretch of residues or overlapping. X₁, X₂, X₃, X₄ and n are as defined above.

Such peptides can be used for the targeting specifically to heart muscle and are more specifically intended for muscular dystrophy, heart diseases or coronary heart diseases. Systemic delivery of vectors targeted with such heart-specific peptides can be considered to avoid regional delivery to the coronary artery that requires an invasive and cumbersome operation. Alternatively, the use of such targeting peptides will limit the spread of vectors after local administration.

A first family relates to a heart targeting peptide comprising at least a three amino acid motif THP or FAT or THP and FAT. Advantageously, it comprises both the THP and FAT motifs, especially when the two motifs are separated by at least one amino acid. Preferably, a heart targeting peptide according to the invention has the sequence:

X₁THPRFATX₂,

X₁RTPFATYX₂, or

X₁FHVNPTSPTHPLX₂.

A second family relates to a heart targeting peptide comprising at least a three amino acid motif QTS. Preferably a heart targeting peptide according to the invention has the sequence:

X₁QTSSPTPLSHTQX₂ or

X₁PQTSTLLX₂.

A third family relates to a heart targeting peptide comprising at least a three amino acid motif HLP or SLF or HLP and SLF. Advantageously, it comprises both the HLP and SLF motifs, especially when the two motifs are separated by at least one amino acid. Preferably, a heart targeting peptide according to the invention has the sequence:

X₁HLPTSSLFDTTHX₂,

X₁VHHLPRTX₂,

X₁QLHNHLPX₂,

X₁HSFDHLPAAALHX₂, or

X₁TRYLPVLPSLFPX₂.

A fourth family relates to a heart targeting peptide comprising at least a three amino acid motif YPS or TNT or YPS and TNT. Advantageously, it comprises both the YPS and TNT motifs, especially when the two motifs are separated by three to eight amino acids. Preferably, a heart targeting peptide according to the invention has the sequence:

X₁YPSAPPQWLTNTX₂,

X₁YPSQSQRX₃LSX₄HX₂,

X₁TYPSSTLX₂,

X₁NTLQVRGVYPSVX₂,

X₁YSNRTNTNSHWAX₂, or

X₁PATNTSKX₂.

A fifth family relates to a heart targeting peptide comprising at least a three amino acid motif HVN or NKL or HVN and NKL. Advantageously, it comprises both HVN and NKL motifs, especially when the two motifs are overlapping. Preferably, a heart targeting peptide of the invention has the sequence:

X₁HVNKLHGX₂,

X₁FHVNPTSPTHPLX₂, or

X₁NANKLWTWVSSPX₂.

A sixth family relates to a heart targeting peptide comprising at least a three amino acid motif SGR. Preferably, a heart targeting peptide according to the invention has the sequence:

X₁SGRIPYLX₂, or

X₁NEDINDVSGRLSX₂.

A seventh family relates to a heart targeting peptide comprising at least a three amino acid motif SPQ, QRA, QRL or PQR or any combination thereof. Advantageously, it comprises the three motifs SPQ, QRA and QRL, especially when the SPQ and QRA motifs are overlapping and separated from the QRL motif by at least one amino acid. Preferably, a heart targeting peptide according to the present invention has the sequence:

X₁LSPQRASQRLYSX₂,

X₁SFSTSPQX₂,

X₁ERMDSPQX₂,

X₁WKSELPVQRARFX₂,

X₁HHGHSPTSPQVRX₂,

X₁GSSTGPQRLHVPX₂,

X₁YPSQSQRX₃LSX₄HX₂,

X₁TCSLCNPVQPQRX₂,

X₁QRLTTLYX₂, or

X₁WSPGQQRLHNSTX₂.

An eighth family relates to a heart targeting peptide comprising at least a three amino acid motif SEL or PVQ or SEL and PVQ. Advantageously, it comprises both the SEL and PVQ motifs, especially when the two motifs are continuous. Preferably, a heart targeting peptide according to the invention has the sequence:

X₁WKSELPVQRARFX₂,

X₁SELPSMRLYTQPX₂,

X₁HSLHVHKGLSELX₂,

X₁SDLPVQLEPERQX₂,

X₁TCSLCNPVQPQRX₂, or

X₁WEPPVQSAWQLSX₂.

A ninth family relates to a heart targeting peptide comprising at least a three amino acid motif QPP or PRP or QPP and PRP. Advantageously, it comprises both the QPP and PRP motifs, especially when the two motifs are continuous. Preferably, a heart targeting peptide according to the invention has the sequence:

X₁HFTFPQQQPPRPX₂,

X₁GSTSRPQPPSTVX₂,

X₁NFSQPPSKHTRSX₂,

X₁QYPHKYTLQPPKX₂,

X₁FNQPPSWRVSNSX₂,

X₁SVSVGMKPSPRPX₂, or

X₁STPRPPLGIPAQX₂.

Heart targeting peptides of the invention are more advantageously intended for targeting any heart cells including the heart vasculature, especially endothelial cells, and heart muscle cells.

According to a fourth embodiment, the present invention provides tumor targeting peptides. A tumor targeting peptide according to the invention has a minimal size of 7 amino acids. Such peptides can be classified in different families that are defined according to the presence of some common amino acid motifs. Each peptide in a family contains a particular motif but in a different amino acid environment. The present invention also encompasses the case where a particular peptide comprises more than one selected motif, that can be continous, separated by a stretch of residues or overlapping. X₁, X₂ and n are as defined above.

Coupling of these peptides to plasmids, viral and synthetic vectors will, for example, allows after systemic administration the targeting of tumor metastasis or tumor sites that are difficult to reach surgically. Alternatively, local administration can also be envisaged with the advantage of limiting the spread of vectors.

A first family relates to a tumor targeting peptide comprising at least a three amino acid motif RPA, NYR or QSP or any combination thereof. Advantageously, it comprises the three motifs RPA, NYR and QSP, especially when the QSP motif is separated from the NYR motif by at least one amino acid and the NYR and RPA motifs are overlapping. Preferably, a tumor targeting peptide according to the invention has the sequence:

X₁TQSPLNYRPALLX₂,

X₁AQSPTIKLTPSWX₂,

X₁TLVQSPMX₂,

X₁NLNTDNYRQLRHX₂,

X₁FRPAVHNMPSLQX₂, or

X₁ISRPAPISVDMKX₂.

A second family relates to a tumor targeting peptide comprising at least a three amino acid motif THR or SRA or THR and SRA. Advantageously, it comprises both the THR and SRA motifs, especially when the two motifs are separated by four to eight amino acids. Preferably, a tumor targeting peptide according to the invention has the sequence:

X₁THRPSLPDSSRAX₂,

X₁ALHPLTHRHYATX₂,

X₁THRGPQSX₂,

X₁SFHMPSRAVSLSX₂, or

X₁NQSNFTSRALLYX₂.

A third family relates to a tumor targeting peptide comprising at least a three amino acid motif PTH, VSP or a four amino acid motif HHVS or any combination thereof. Advantageously, it comprises the three motifs PTH, HHVS and VSP, especially when the PTH motif is separated from the HHVS motif by at least one amino acid and the HHVS and VSP motifs are overlapping. Preferably, a tumor targeting peptide according to the invention has the sequence:

X₁SFPTHIDHHVSPX₂,

X₁LNGDPTHX₂,

X₁HMPHHVSNLQLHX₂ or

X₁LPSVSPVLQVLGX₂.

A fourth family relates to a tumor targeting peptide comprising at least a three amino acid motif YLS or QQL or YLS and QQL. Advantageously, it comprises both YLS and QQL motifs, especially when the two motifs are continous. Preferably a tumor targeting peptide according to the invention has the sequence:

X₁DAQQLYLSNWRSX₂,

X₁DSYLSSTLPGQLX₂ or

X₁SPTPTSHQQLHSX₂.

A fifth family relates to a tumor-targeting peptide comprising at least a three amino acid motif SND or SAI or SND and SAI. Advantageously, it comprises both the SND and SAI motifs, especially when the two motifs are continous. Preferably, a tumor targeting peptide according to the invention has the sequence:

X₁MHNVSDSNDSAIX₂,

X₁DNSNDLMX₂, or

X₁TVMEAPRSAILIX₂.

A sixth family relates to a tumor-targeting peptide comprising at least a three amino acid motif NDI, WPY, MPL, PSH, LPQ, WPV or WPT or any combination thereof. According to a special embodiment, said sixth family relates to a said peptide comprising at least one amino acid motif WPX₃X₄PW, with X₃ and X₄, identical or different, represent any amino acid; preferably X₃ is V or T and/or X₄ is R or S. In another special embodiment, said peptide comprises at least one amino acid motif WPTSPWX₃X₄RX₅ with X₃ ,X₄ and X₅, identical or different, represent any amino acid; preferably X₃ is L or S and/or X₄ is E or S and/or X₅ is E or D. In another special embodiment, said peptide comprises at least one amino acid motif WPX₃X₄SX₅F with X₃,X₄ and X₅, identical or different, represent any amino acid; preferably X₃ is Y or M and/or X₄ is P or K and/or X₅ is L, Q or H. Preferably, a tumor targeting peptide according to the invention has the sequence:

X₁CNDIGWVRCX₂,

X₁CWPYPSHFCX₂,

X₁MPLPQPSHLPLLX₂,

X₁LPQRAFWVPPIVX₂,

X₁WPVRPWMPGPVVX₂,

X₁WPTSPWLEREPAX₂, or

X₁WPTSPWSSRDWSX₂.

A seventh family relates to a tumor-targeting peptide comprising at least a three amino acid motif HEW, QID, WPM or CLP or any combination thereof. Preferably, a tumor targeting peptide according to the invention has the sequence:

X₁HEWSYLAPYPWFX₂

X₁QIDRWFDAVQWLX₂

X₁CLPSTRWTCX₂, or

X₁CWPMKSX₅FCX2

Preferably, X₅ is a leucine (L) or a glutamine (Q) residue.

Advantageously, a tumor targeting peptide of the present invention may be used for the targeting of a therapeutic agent to a tumor cell, a metastasis or a tumor vasculature.

According to a fifth embodiment, the present invention provides for a composition comprising at least one peptide according to the present invention and at least one therapeutic agent or alternatively at least one nucleic acid molecule encoding a peptide of the invention and at least one therapeutic agent.

As used herein, a <<therapeutic agent>> is used broadly to mean an organic chemical such as a drug (i.e. a cytotoxic drug), a peptide including a variant or a modified peptide or a peptide-like molecule, a protein, an antibody or a fragment thererof such as a Fab (ab for antigen binding), a F(ab′)2, a Fc (c for crystallisable) or a scFv (sc for single chain and v for variable). Antibody fragments are described in detail in immunology manuals (such as Immunology, third edition 1993, Roitt, Brostoff and Male, ed Gambli, Mosby). It is also possible to use a chimeric antibody or protein derived from the sequence of diverse origins. As an example, humanized antibodies combine part of the variable regions of a mouse antibody and constant regions of a human immunoglobulin. Within the context of the present invention, a protein is more preferably an immunostimulatory protein, such as B7.1, B7.2, CD40, ICAM, CD4, CD8 and the like. A therapeutic agent may also be a nucleic acid molecule e.g. DNA, or RNA, antisense or sense, oligonucleotide, double-stranded or single-stranded, circular or linear . . . etc.

In a preferred embodiment, a therapeutic agent is a vector for delivering at least one therapeutic gene or gene of interest to a target cell of a vertebrate. In the context of the present invention, it can be a plasmid, a synthetic (non viral) or a viral vector.

Plasmid denotes an extrachromosomic circular DNA capable of autonomous replication in a given cell. The choice of the plasmids is very large. It is preferably designed for amplification in bacteria and expression in eukaryotic host cell. Such plasmids can be purchased from a variety of manufacturers. Suitable plasmids include but are not limited to those derived from pBR322 (Gibco BRL), pUC (Gibco BRL), pBluescript (Stratagene), pREP4, pCEP4 (Invitrogene), pCI (Promega) and p Poly (Lathe et al., Gene 57 (1987), 193-201). It is also possible to engineer such a plasmid by molecular biology techniques (Sambrook et al., Laboratory Manual, Cold (Spring Harbor Laboratory Press, Cold Spring Harbor (1989), N.Y.). A plasmid may also comprise a selection gene in order to select or identify the transfected cells (e.g. by complementation of a cell auxotrophy, antibiotic resistance), stabilizing elements (e.g. cer sequence; Summers and Sherrat, Cell 36 (1984), 1097-1103) or integrative elements (e.g. LTR viral sequences).

A vector may also be from viral origin and may be derived from a variety of viruses, such as herpes viruses, cytomegaloviruses, foamy viruses, lentiviruses, AAV (adeno-associated virus), poxviruses, adenoviruses and retroviruses. Such viral vectors are well known in the art. The term <<viral vector>> as used in the present invention encompasses the vector genome, the viral particles (i.e. the viral capsid including the viral genome) as well as empty viral capsids.

A viral vector which is particularly appropriate for the present invention is an adenoviral vector (for a review see for example Hitt et al. Advances in Pharmacology 40 (1997) 137-206). In one embodiment, the adenoviral vector is engineered to be conditionally replicative (CRAd vectors) in order to replicate selectively in specific cells (e.g. proliferative cells) as described in Heise and Kirn (2000, J. Clin. Invest. 105, 847-851). According to a second and preferred alternative, it is replication-defective, especially for E1 functions by total or partial deletion of the respective region. Advantageously, the E1 deletion covers nucleotides (nt) 458 to 3510 by reference to the sequence of the human adenovirus type 5 disclosed in the Genebank data base under the reference M 73260. Furthermore, the adenoviral backbone of the vector may comprise additional modifications, such as deletions, insertions or mutations in one or more viral genes. An example of an E2 modification is illustrated by the thermosensible mutation located on the DBP (DNA Binding Protein) encoding gene (Ensinger et al., J. Virol. 10 (1972), 328-339). The adenoviral sequence may also be deleted of all or part of the E4 region. A partial deletion retaining the ORFs 3 and 4 or ORFs 3 and 6/7 may be advantageous (see for example European patent application EP98401722.8). In addition, the adenoviral vector in use in the present invention may be deleted of all or part of the E3 region. In this context, it might be interesting to retain the E3 sequences coding for the polypeptides allowing to escape the host immune system (Gooding et al., Critical Review of Immunology 10 (1990), 53-71). A defective adenoviral vector deficient in all early and late regions may also be envisaged.

The adenoviral vector in use in the present invention may be derived from a human or animal adenovirus genome, in particular a canine, avian, bovine, murine, ovine, feline, porcine or simian adenovirus or alternatively from a hybrid thereof. Any serotype can be employed. One can cite in particular the canine CAV-1 or CAV-2 adenovirus (Genbank ref CAV1GENOM and CAV77082 respectively), the avian adenovirus (Genbank ref AAVEDSDNA), the mouse adenovirus (Genbank ref ADRMUSMAV1) and the bovine BAV3 (Seshidhar Reddy et al. J. Virol. 72 (1998) 1394-1402). However, the human adenoviruses of C sub-group are preferred and especially adenoviruses 2 (Ad2) and 5 (Ad5). Generally speaking, the cited viruses are available in collections such as ATCC and have been the subject of numerous publications describing their sequence, organization and biology, allowing the artisan to practice them.

The recombinant adenoviral vector is packaged to constitute infectious virions capable of infecting target cells and transferring the therapeutic gene. Infectious adenoviral particles may be prepared according to any conventional technique in the field of the art (e.g. cotransfection of suitable adenoviral fragments in a 293 cell line as described in Graham and Prevect, Methods in Molecular Biology, Vol 7 (1991), Gene Transfer and Expression Protocols; Ed E. J. Murray, The Human Press Inc, Clinton, N.J.; homologous recombination as described in WO96/17070). The defective virions are usually propagated in a complementation cell line or via a helper virus, which supplies in trans the non functional viral genes. The cell line 293 is commonly used to complement the E1 function (Graham et al., J. Gen. Virol. 36 (1977), 59-72). Other cell lines have been engineered to complement doubly defective vectors (Yeh et al. J. Virol. 70 (1996), 559-565; Krougliak and Graham, Human Gene Ther. 6 (1995), 1575-1586; Wang et al., Gene Ther. 2 (1995), 775-783; Lusky et al., J. Virol. 72 (1998), 2022-2033; WO94/28152 and WO97/04119). The infectious viral particles may be recovered from the culture supernatant but also from the cells after lysis and optionally further purified according to standard techniques (chromatography, ultracentrifugation in a cesium chloride gradient . . . ).

In addition, adenoviral virions or empty adenoviral capsids can also be used to transfer nucleic acids (i.e. plasmidic vectors) by a virus-mediated cointernalization process as described in U.S. Pat. No. 5,928,944. This process can be accomplished in the presence of a cationic agent(s) such as polycarbenes or lipoplex vesicles comprising one or more lipid layers.

A retroviral vector is also suitable. The numerous vectors described in the literature may be used within the framework of the present invention and especially those derived from murine leukemia viruses (i.e. Moloney or Friend's). Generally, a retroviral vector is deleted of all or part of the viral genes gag, pol and env and comprises 5′LTR, an encapsidation sequence and 3′LTR. These elements may be modified to increase expression level or stability of the retroviral vector. The therapeutic gene is preferably inserted downstream of the encapsidation sequence. The propagation of such a vector requires the use of complementation lines as described in the prior art.

A poxviral vector may be derived e.g. from an avian poxvirus such as the canarypox, a fowlpox virus or a vaccinia virus, the latter being preferred. Among all the vaccinia viruses which can be envisaged within the framework of the present invention, the Copenhagen, Wyeth and modified Ankara (MVA) strains are preferably chosen. The general conditions for obtaining a vaccinia virus capable of expressing a therapeutic gene are disclosed in European patent EP 83 286 and application EP 206 920. MVA viruses are more particularly described in Mayr et al. (Infection 3 (1975) 6-14) and Sutter and Moss (Proc. Natl. Acad. Sci. USA 89 (1992) 10847-10851).

According to another alternative, a therapeutic agent also refers to a non viral (synthetic) vector that is capable to deliver a therapeutic gene to a target cell, for example lipoplexes. Lipoplexes may contain cationic lipids which have a high affinity for nucleic acids and interact with the cell membranes (Felgner et al. Nature 337 (1989) 387-388). As a result, they are capable of complexing the nucleic acid, thus generating a compact particle capable to enter the cells. Many laboratories have already disclosed various lipoplexes. By way of examples, there may be mentioned DOTMA (Felgner et al., Proc. Natl. Acad. Sci. USA 84 (1987), 7413-7417), DOGS or Transfectam™ (Behr et al., Proc. Natl. Acad. Sci. USA 86 (1989), 6982-6986), DMRIE or DORIE (Felgner et al., Methods 5 (1993), 67-75), DC-CHOL (Gao and Huang, BBRC 179 (1991), 280-285), DOTAP™ (McLachlan et al., Gene Therapy 2 (1995), 674-622), Lipofectamine™ and glycerolipid compounds (see WO98/34910 and WO98/37916).

Other non viral (synthetic) vectors have been developed which are based on cationic polymers such as polyamidoamine (Haensler and Szoka, Bioconjugate Chem. 4 (1993), 372-379), dendritic polymer (WO 95/24221), polyethylene imine or polypropylene imine (WO 96/02655), polylysine (U.S. Pat. No. 5,595,897 or FR 2 719 316), chitosan (U.S. Pat. No. 5,744,166) or DEAE dextran (Lopata et al. Nucleic Acid Res. 12 (1984) 5707-5717).

The term “therapeutic gene or gene of interest” refers to a nucleic acid (DNA, RNA or other polynucleotide derivatives). It can code, e.g., for an antisense RNA, a ribozyme or a messenger (mRNA) that will be translated into a polypeptide. It includes genomic DNA, cDNA or mixed types (minigene). It may code for a mature polypeptide, a precursor (e.g. a precursor comprising a signal sequence intended to be secreted or a precursor intended to be further processed by proteolytic cleavage . . . ), a truncated polypeptide or a chimeric polypeptide. The gene may be isolated from any organism or cell by the conventional techniques of molecular biology (PCR, cloning with appropriate probes, chemical synthesis) and if needed its sequence may be modified by mutagenesis, PCR or any other protocol.

The following genes are of particular interest. For example genes coding for a cytokine (α, β or γ interferon, interleukine (IL), in particular IL-2, IL-6, IL-10 or IL-12, a tumor necrosis factor (TNF), a colony stimulating factor GM-CSF, C-CSF, M-CSF . . . ), a immunostimulatory polypeptide (B7.1, B7.2, CD40, CD4, CD8, ICAM and the like), a cell or nuclear receptor, a receptor ligand (fas ligand), a coagulation factor (FVIII, FIX . . . ), a growth factor (Transforming Growth Factor TGF, Fibroblast Growth Factor FGF and the like), an enzyme (urease, renin, thrombin, metalloproteinase, nitric oxide synthase NOS, SOD, catalase . . . ), an enzyme inhibitor (α1-antitrypsine, antithrombine III, viral protease inhibitor, plasminogen activator inhibitor PAI-1), the CFTR protein, insulin, dystrophin, a MHC antigen (Major Histocompatibility Complex) of class I or II or a polypeptide that can modulate/regulate expression of cellular genes, a polypeptide capable of inhibiting a bacterial, parasitic or viral infection or its development (antigenic polypeptides, antigenic epitopes, transdominant variants inhibiting the action of a native protein by competition . . . ), an apoptosis inducer or inhibitor (Bax, Bcl2, BclX . . . ), a cytostatic agent (p21, p 16, Rb . . . ), an apolipoprotein (ApoAI, ApoAIV, ApoE . . . ), an inhibitor of angiogenesis (angiostatin, endostatin . . . ), an angiogenic polypeptide (family of Vascular Endothelial Growth Factors VEGF, FGF family, CCN family including CTGF, Cyr61 and Nov), an oxygen radical scaveyer, a polypeptide having an anti-tumor effect, an antibody, a toxin, an immunotoxin and a marker (β-galactosidase, luciferase . . . ) or any other genes of interest that are recognized in the art as being useful for the treatment or prevention of a clinical condition.

In view of treating an hereditary dysfunction, one may use a functional allele of a defective gene, for example a gene encoding factor VII ou IX in the context of haemophilia A or B, dystrophin (or minidystrophin) in the context of myopathies, insulin in the context of diabetes, CFTR (Cystic Fibrosis Transmembrane Conductance Regulator) in the context of cystic fibrosis. Suitable anti-tumor genes include but are not limited to those encoding an antisense RNA, a ribozyme, a cytotoxic product such as thymidine kinase of herpes-1 simplex virus (TK-HSV-1), ricin, a bacterial toxin, the expression product of yeast genes FCY1 and/or FUR1 having UPRTase (Uracile Phosphoribosyltransferase) and CDase (Cytosine Deaminase) activities, an antibody, a polypeptide inhibiting cellular division or transduction signals, a tumor suppressor gene (p53, Rb, p73 . . . ), a polypeptide activating host immune system, a tumor-associated antigen (MUC-1, BRCA-1, an HPV early or/and late antigen (E6, E7, L1, L2 . . . ) . . . ), optionally in combination with a cytokine gene.

The therapeutic gene may be engineered as a functional expression cassette, including a suitable promoter. The latter may be obtained from any viral, prokaryotic, e.g. bacterial, or eukaryotic gene (even from the gene of interest), be constitutive or regulable. Optionally, it may be modified in order to improve its transcriptional activity, delete negative sequences, modify its regulation, introduce appropriate restriction sites etc. Suitable promoters include but are not limited to adenoviral E1a, MLP, PGK (Phospho Glycero Kinase; Adra et al. Gene 60 (1987) 65-74; Hitzman et al. Science 219 (1983) 620-625), MT (metallothioneine; Mc Ivor et al., Mol. Cell Biol. 7 (1987), 838-848), α-1 antitrypsin, CFTR, surfactant, immunoglobulin, β-actin (Tabin et al., Mol. Cell Biol. 2 (1982), 426-436), SRα (Takebe et al., Mol. Cell. Biol. 8 (1988), 466-472), early SV40 (Simian Virus), RSV (Rous Sarcoma Virus) LTR, TK-HSV-1, SM22 (WO 97/38974), Desmin (WO 96/26284) and early CMV (Cytomegalovirus; Boshart et al. Cell 41 (1985) 521). Alternatively, promoters can be used which are active in tumor cells. Suitable examples include but are not limited to the promoters isolated from MUC-1 gene overexpressed in breast and prostate cancers (Chen et al., J. Clin. Invest. 96 (1995), 2775-2782), CEA (Carcinoma Embryonic Antigen) overexpressed in colon cancers (Schrewe et al., Mol. Cell. Biol. 10 (1990), 2738-2748), tyrosinase overexpressed in melanomas (Vile et al., Cancer Res. 53 (1993), 3860-3864), ErbB-2 overexpressed in breast and pancreas cancers (Harris et al., Gene Therapy 1 (1994), 170-175) and ═-foetoprotein overexpressed in liver cancers (Kanai et al., Cancer Res. 57 (1997), 461-465). The early CMV promoter is preferred in the context of the invention.

The expression cassette may further include additional functional elements, such as intron(s), secretion signal, nuclear localization signal, IRES, poly A transcription termination sequences, tripartite leader sequences and replication origins.

The vector in use in the present invention may comprise one or more gene(s) of interest. The different genes may be included in the same cassette or in different cassettes thus controled by separate regulatory elements. The cassettes may be inserted into various sites within the vector in the same or opposite directions. According to another alternative, the different genes may be placed on different vectors.

Optionally, a therapeutic agent in use in the present invention can be associated with one or more stabilizing substance(s) such as lipids (i.e. cationic lipids such as those described in WO98/44143, liposomes), nuclease inhibitors, polymers, chelating agents in order to prevent degradation within the human/animal body.

According to a preferred embodiment, the peptide of the present invention is operably coupled to the therapeutic agent. “Operably coupled” means that the components so described are in a relationship permitting them to function in their intended manner (i.e. the peptide promotes the targeting of the therapeutic agent to the desired cell). The coupling can be made by different means that are well known to those skilled in the art and include covalent, non covalent or genetic means.

Covalent attachment of peptides to the surface of the therapeutic agent may be performed through reactive functional groups at the surface of the therapeutic agent, optionally with the intermediary use of a cross linker or other activating agent (see for example Bioconjugate techniques 1996; ed G Hermanson; Academic Press). The functional groups of the therapeutic agent may be modified to be reactive towards specific amino acid groups of the peptide. In particular, coupling may be done with (i) homobifunctional or (ii) heterobifunctional cross-linking reagents, with (iii) carbodiimides, (iv) by reductive amination or (vi) by activation of carboxylates.

Homobifunctional cross linkers including glutaraldehyde and bis-imidoester like DMS (dimethyl suberimidate) can be used to couple amine groups of peptides to lipoplexes containing diacyl amines such as phosphatidylethanolamine (PE) residues. Other examples are given in Bioconjugate techniques (1996) 188-228; ed G Hermanson; Academic Press).

Many heterobifunctional cross linkers can be used in the present invention, in particular those having both amine reactive and sulfhydryl-reactive groups, carbonyl-reactive and sulfhydryl-reactive groups and sulfhydryl-reactive groups and photoreactive linkers. Suitable heterobifunctional crosslinkers are described in Bioconjugate techniques (1996) 229-285; ed G Hermanson; Academic Press) and WO99/40214. Examples of the first category include but are not limited to SPDP (N-succinimidyl 3-(2-pyridyidithio) propionate), SMBP (succinimidyl-4-(p-maleimidophenyl) butyrate), SMPT (succinimidyloxycarbonyl-α-methyl-(α-2-pyridyldithio) toluene), MBS (m-maleimidobenzoyl-N-hydroxysuccinimide ester), SIAB (N-succinimidyl (4 iodoacetyl) aminobenzoate), GMBS (γ-maleimidobutyryloxy) succinimide ester), SIAX (succinimidyl-6-iodoacetyl amino hexonate, SIAC (succinimidyl-4-iodoacetyl amino methyl), NPIA (p-nitrophenyl iodoacetate). The second category is useful to couple carbohydrate-containing molecules (e.g. env glycoproteins, antibodies) to sulfydryl-reactive groups. Examples include MPBH (4-(4-N maleimidophenyl) butyric acid hydrazide) and PDPH (4-(N-maleimidomethyl) cyclohexane-1-carboxyl-hydrazide (M₂C₂H and 3-2(2-pyridyldithio) proprionyl hydrazide). As an example of the third category, one may cite ASIB (1-(p azidosalicylamido)-4-(iodoacetamido) butyrate). Another alternative includes the thiol reactive reagents described in Frisch et al. (Bioconjugate Chem. 7 (1996) 180-186).

Coupling (iii) involves, e.g., amine groups of underivatized PE present in lipoplexes that can participate in the carbodiimide reaction with carboxylate groups on proteins.

Coupling (iv) may be performed, e.g., via imine formation followed by reduction using a cyanoborohydrate.

Coupling (vi) may involve, e.g., an NHS ester derivative of lipoplexe and a peptide amine group to produce stable amide bond linkages.

Another example uses a maleimide-sulfhydryl bond involving a sulfhydryl group and a sulfhydryl reactive group. For example SATA (N-succinimidyl S-acelythioacetate) can be used to introduce a sulfhydryl group whereas sulfo SMCC (sulfosuccinimidyl 4-(N-maleimidomethyl) cyclo hexane 1-carboxylate) can be used to introduce a maleimide group resulting in a covalent thioether bond.

Another preferred linker is a polymer such as polyethylene glycol (PEG) or its derivatives. Preferably, such a polymer has an average molecular weight comprised between 200 to 20000 Da. For example, tresyl-MPEG can be used to couple an ε amino group present on Lys residues (see for example WO99/40214). Other means to conjugate two partners via PEG are described in the literature (in Bioconjugate techniques (1996) 606-618; ed G Hermanson; Academic Press and Frisch et al. Bioconjugate Chem. 7 (1996) 180-186).

Non covalent coupling includes electrostatic interactions, for example between a cationic peptide and a negatively charged plasmidic or viral vector or between an anionic peptide and a cationic synthetic vector. Another alternative consists in using affinity components such as Protein A, biotin/avidin, antibodies, which are able to associate non covalently or by affinity on the one hand the peptide of the invention and on the other hand the therapeutic agent. Concerning lipoplexes, biotinylated PE derivatives can be used to interact non covalently with avidin peptide conjugates or with other biotinylated peptides using avidin as a bridging molecule (Bioconjugate techniques (1996) 570-591; ed G Hermanson; Academic Press). Coupling with viral vectors may use biotinylated antibodies directed against a capsid epitope and streptavidin-labelled antibodies directed against a peptide of the invention (Roux et al. Proc. Natl. Acad Sci USA 86 (1989) 9079).

Covalent coupling with plasmidic vectors may use an alkylating agent (Sebestyen et al. Nat. Biotechnol. 16 (1998) 80-85; Ciolina et al. Bioconjug. Chem. 10 (1999) 49-55; Zanta et al. Proc. Natl. Acad. Sci. USA 96 (1999) 91-96). Non covalent coupling may be achieved by using PNA (Peptide Nucleic Acid) or triple helix (Neves et al. Cell Biol. Toxicol. 15 (1999) 193-202; Neves et al. FEBS Lett. 453 (1999) 41-45) or by any coupling agent interacting with nucleic acids, such as anti DNA immunoglobulins as described in WO97/02840 or polycationic compounds such as polylysine. Bifunctional antibodies directed against each of the coupling partners are also suitable for this purpose.

Genetic coupling is more particularly intended for coupling a peptide according to the invention and a viral vector. Advantageously, a nucleic acid encoding such a peptide can be inserted in addition or in place of a native viral sequence that encodes a polypeptide exposed at the viral surface, to make the peptide of the invention expressed at the surface of the virus particle. Insertion sites can be selected on the basis of three-dimensional data in order to identify regions that are non essential for virus integrity. Suitable surface exposed polypeptides include the envelope protein of a retroviral vector and the adenoviral capsid proteins, such as fiber, hexon and penton base. In the context of the present invention, insertion into the fiber gene is preferred (Ad2 fiber gene described in Herissé et al; Nucleic Acid Res. 9 (1981) 4023-4042; Ad5 fiber gene described in Chroboczek et al. Virol. 161 (1987) 549-554). Preferably, the sequences that ensure proper trimerization and association with the penton base complex are preserved whereas those coding for the CAR binding-site (Roelvink et al. Science 286 (1999) 1568-1571) are altered. Insertion in different loops of the knob domain, more specifically in AB, CD, DG, GH and IJ loops, or just upstream to the STOP codon can be envisaged. Examples of appropriate locations are illustrated in WO94/10323, WO95/26412, WO95/05201, WO96/26281, WO98/44121 and FR99 10859. Introduction of the peptide encoding DNA in the penton base encoding sequence may be performed as described in WO96/07734 and U.S. Pat. No. 5,559,099.

Alternatively, coupling between the peptide of the invention and the therapeutic agent may be done in the organism at the site of the cells to be targeted. According to such an embodiment, non covalent coupling is preferred. For example, one may envisage to introduce in the organism or to the target cell (i) the peptide according to the invention associated with a first affinity component (e.g. biotin) and (ii) the therapeutic agent associated with a second affinity component capable to bind the first one (e.g. avidin). Preferably, (i) is introduced before (ii).

As indicated before, the composition of the present invention may comprise a nucleic acid encoding the peptide of the invention instead of the peptide as such. According to a first alternative, the nucleic acid encoding such a peptide can be fused to a therapeutic gene. The fusion sequence can be placed under the control of suitable elements allowing its expression (e.g. a promoter) and incorporated in a conventional vector which can be introduced into an organism to be treated in order to locally express a fusion polypeptide that combines both targeting and therapeutic properties. A preferred fusion sequence is obtained by fusing the nucleic acid encoding a tumor-targeting peptide of the present invention and an immunostimulatory gene (e.g. B7.1) and is engineered to include functional elements allowing secretion of the fusion polypeptide outside the expressing cells (presence of a signal sequence). Injection of such a fusion sequence to an organism having cancer will result in the synthesis and secretion of a fusion polypeptide allowing the targeting of the tumor cells present in the organism and the in situ delivery of the immunostimulatory polypeptide capable of enhancing the anti-tumoral response. Another alternative would be to incorporate into two adenoviral particles on the one hand genes encoding retroviral helper functions (gag/pol and env genes) with the env gene comprising a nucleic acid encoding a peptide according to the invention and on the other hand a conventional retroviral vector engineered to express a therapeutic gene. Cells co-infected with the two adenoviral particles will produce infectious retroviral particles with an envelope exposing the targeting peptide. The use of a tumor-targeting peptide will allow local targeting of tumoral cells.

In accordance with the goal pursued by the present invention, the peptide and/or the therapeutic agent may be modified to improve or stabilize the coupling. In particular, the peptide may be extended by a spacer at the N or C-terminus to facilitate its accessibility to target cells after coupling.

Moreover, a composition according to the invention may comprise one or more peptides of the invention that may or may not be fused (i.e. in tandem). For example, when it is desirable to enhance the specificity of the composition of the invention towards a specific target, it may be advantageous to use a combination of targeting peptides.

A composition according to the invention may be manufactured in a conventional manner for local, systemic, oral, rectal or topical administration. Suitable routes of administration include but are not limited to intragastric, subcutaneous, aerosol, instillation, inhalation, intracardiac, intramuscular, intravenous, intraarterial, intraperitoneal, intratumoral, intranasal, intrapulmonary or intratracheal routes. The administration may take place in a single dose or a dose repeated one or several times after a certain time interval. The appropriate administration route and dosage vary in accordance with various parameters, for example, with the individual, the disorder to be treated, the therapeutic agent or with the gene of interest to be transferred. As far as viral vectors are concerned, the corresponding viral particles may be formulated in the form of doses of between 10⁴ and 10¹⁴ iu (infectious unit), advantageously between 10⁵ and 10¹³ iu and preferably between 10⁶ and 10¹² iu. The titer may be determined by conventional techniques (see for example Lusky et al., 1998, supra). Doses based on a plasmid or synthetic vector may comprise between 0.01 and 100 mg of DNA, advantageously between 0.05 and 10 mg and preferably between 0.5 and 5 mg. The formulation may also include a pharmaceutically acceptable diluent, adjuvant, carrier or excipient. In addition, a composition according to the present invention may include buffering solutions, stabilizing agents or preservatives adapted to the administration route. For example, an injectable solution may be liquid or in the form of a dry powder (lyophylized . . . etc) that can be reconstituted before use. Compositions for topical administration may be in the form of creams, ointments, lotions, solutions or gels. Compositions for intrapulmonary administration may be in the form of powder, spray or aerosol.

A composition according to the present invention can be administered directly in vivo by any conventional and physiologically acceptable administration route, for example by intraarterial injection, into an accessible tumor, into the lungs by means of an aerosol or instillation, into the vascular system using an appropriate catheter, etc. The ex vivo approach may also be adopted which consists in removing cells from the patient (bone marrow cells, peripheral blood lymphocytes, myoblasts and the like . . . ), introducing the composition of the invention in accordance with the techniques of the art and readministering them to the patient. As mentioned above, administration may be performed according to a two steps procedure, the first step consisting of administering a peptide of the invention associated with a first affinity component in order to target the desired cells and the second step consisting of administering the therapeutic agent associated with a second affinity component capable of binding the first one.

Finally, the present invention also provides for the use of a composition according to the invention, for the preparation of a drug intended for gene transfer and preferably for the treatment of human or animal body by gene therapy. Within the meaning of the present invention, gene therapy has to be understood as a method for introducing any therapeutic gene into a cell. Thus, it also includes immunotherapy that relates to the introduction of a potentially antigenic epitope into a cell to induce an immune response which can be cellular or humoral or both. The use of a composition according to the invention is dependent upon the targeting properties of the peptide included in said composition. A composition comprising a heart targeting peptide is preferably used for the treatment or prevention of any disease affecting the heart or its vasculature, such as coronary heart diseases, heart failure, heart hypertrophy, infarction, myocarditis, ischemia, restenosis, atherosclerosis, muscular and the like. A preferred use for a composition comprising a tumor targeting peptide consists in treating or preventing cancers, tumors and diseases which result from unwanted cell proliferation. One may cite more particularly cancers of breast, uterus (in particular, those induced by a papilloma virus), prostate, lung, bladder, liver, colorectal, pancreas, stomach, esophagus, larynx, central nervous system, blood (lymphomas, leukemia, etc.), melanomas and mastocytoma.

The present invention also relates to a method of treatment in which a therapeutically effective amount of a peptide or a composition according to the invention is administered to a patient in need of such a treatment. <<Treatment >> as used herein refers to prophylaxis and therapy. A <<therapeutically effective amount of a peptide or a composition >> is a dose sufficient to the alleviation of one or more symptoms normally associated with the disease desired to be treated. A method according to the invention is more intended for the treatment of the diseases listed above.

The disclosure of all patents, publications including published patent applications, and database entries cited in the present application are hereby incorporated by reference in their entirety to the same extend as if each such individual patent, publication and database entry were specifically and individually indicated to be incorporated by reference and were set forth in its entirety herein.

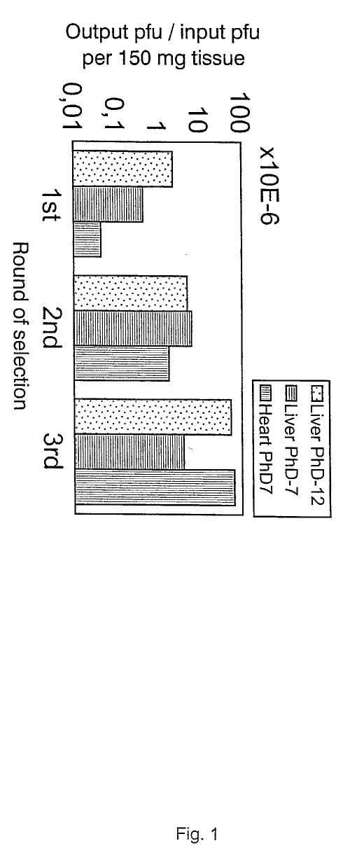

FIG. 1 represents schematically the total number of recovered phages (output pfu (plaque forming units)) calculated per 150 mg organ (liver or heart), for three rounds of in vivo selection with different phage display libraries. All numbers are divided with the titers obtained from the injected inputs to be able to compare between mice.

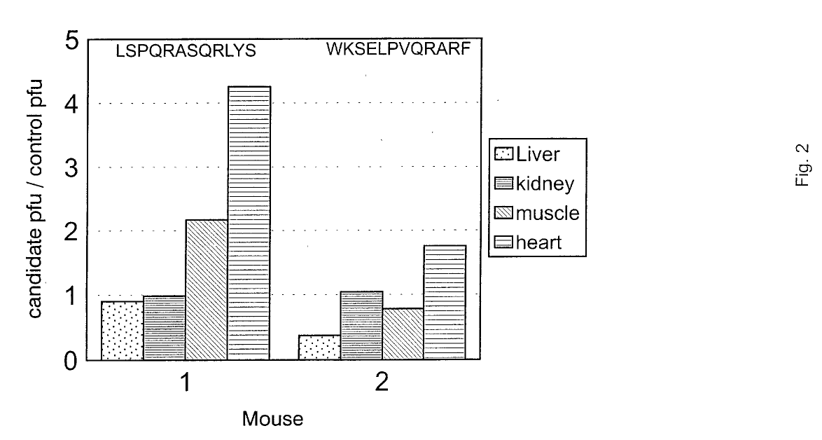

FIG. 2 represents schematically an example of in vivo testing of specificity of candidate phages with a co-injected negative control. The sequence of the displayed peptides is shown in the figure.

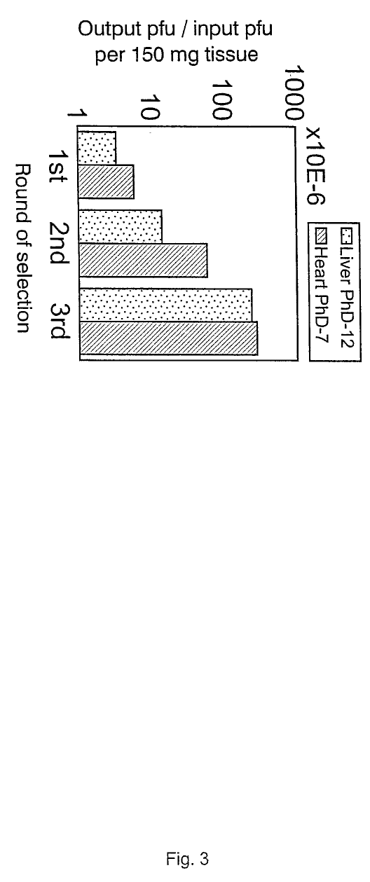

FIG. 3 represents schematically the total number of recovered phages (output pfu) calculated per 150 mg of fixed and minced organ (liver or heart), for three rounds of ex vivo selection with two different phage display libraries. All numbers are divided by the titers obtained from the input amount of phages to be able to compare between mice.

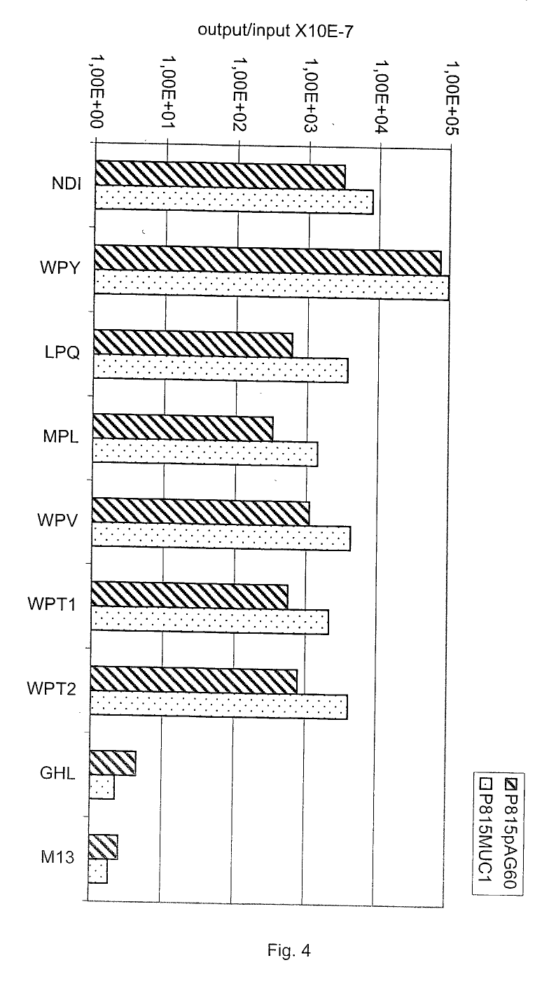

FIG. 4 represents schematically an example of in vitro testing of the specificity of candidate phages binding to P815 cells. The sequence of the displayed peptides is indicated by the three amino acid motif present at the N-terminus. M13 phage and a non-selected phage (GHL) are used as negative controls.

FIG. 5. represents schematically an example of in vitro testing of the specificity of candidate phages binding to WiDr cells in comparison to other cells. The sequence of the displayed peptides is indicated by the three amino acid motif present at the N-terminus. M13 phage and three non-specific phages (not shown) are used as negative controls.

The following examples serve to illustrate the present invention.

Peptides of the invention have been identified using a phage display peptide library. This technology conventional in the domain of the art is detailled in the following documents (Scott et al. Science 249 (1990) 368; Cwirla et al. Proc. Natl. Acad. Sci. USA 87 (1990) 6378; Devlin et al. Science 249 (1990) 404; Romanczuk et al. Hum. Gene Ther. 10 ( 1999) 2615; Samoylova et al. Muscle and Nerve 22 (1999) 460). One of the most commonly used phages for phage display libraries is the filamentous phage M13. The M13 phage can be designed to display on its surface a foreign peptide fused to a coat protein and to harbor the gene for the fusion protein within its genome. The pIII and pVIII surface proteins of the M13 virion are currently used in phage display. The pill protein is present in 3 to 5 copies closely positioned to each other. The pVIII protein is present in about 2700 copies distributed over the surface of the phage. Random peptide sequences can be incorporated at the N-terminus of either proteins.

Different strategies are available to select phages that selectively target a desired cell type. The first screening method involves in vivo injection of the phage display library, isolation of the target tissue and amplification by subjecting the retained phages to two or more rounds of in vivo selection towards the same organ (Rajotte et al. J. Clin. Invest. 102 (1998) 430-437; Pasqualini et al. Nature 380 (1996) 364-366; Pasqualini et al. Nature Biotechnology 15 (1997) 542-546). The in vivo approach is applicable for targeting various tissues (injection in wild type animals), tumors (using tumor animal models) and affected cells (injection in various animal models, for example artherosclerotic plaques in KO mice or ischaemic limbs).

In the second approach (ex vivo), organs or tissues are isolated, cut in small pieces, slightly fixed and then incubated with the phage library. Unbound phages are removed by washing, and bound phages are eluted at low pH or by directly adding host bacteria. The retained phages are amplified in bacteria and then further enriched by reexposure to the target (Van Ewijk et al. Proc. Natl. Acad. Sci. USA 94 (1997) 3903; Odermatt et al. J. Am. Soc. Nephr. 10 (1999) 448). The ex vivo selection scheme allows the use of human samples as the selecting tissues, for example biopsies from heart muscle, tumors or artherosclerotic plaques.

Finally, the in vitro selection strategy is based on the adsorption of phages to cultured cells (Waters et al. Immunotechnology 3 (1997) 21; Barry et al. Nature Medecine 2 (1996) 299; Samoylava et al. Muscle and Nerve 22 (1999) 460). A pre-adsorption step can be realized to eliminate the phages that exhibit a strong unspecific binding, for example those which display long stretches of positively and negatively charged amino acids. For this purpose, the library may be pre-adsorbed to unrelated cell lines (different from the target cells), non transformed cells from the same tissue or plastic surfaces. Then, the phage display library is incubated with the target cells grown in culture. Unbound phages are washed away and bound phages are eluted, recovered and amplified. When tumor targeting is concerned, the in vitro selection is performed on cultured tumor cell lines from various origins or primary tumor cells prepared from tumor tissues. Furthermore, the extracellular matrix (ECM) of tumors represents another potential target. ECM can be isolated from tumors (i.e. matrigel), fixed to tissue cuture dishes and used to select phages. Also, phages can be selected against isolated molecules (Burg et al. Cancer Res. 59 (1999) 2869; Koivuen et al. Nature Biotechn. 17 (1999) 768).

The in vitro selection on cell lines can be extended to select peptides that are specific for certain cell-surface exposed proteins. Several tumor specific cell surface antigens are known and could be used as specific addresses. Some examples are listed in Table 1. In this approach, the cell-surface receptor is expressed in a cell line after stable transformation of appropriate expression plasmids. Phages are first pre-selected against the parental cell line which does not express the receptor and then positively selected on the receptor expressing cells. This allows to select peptides against target proteins which are not available in purified form and has the additional advantage of displaying a receptor in the context of the cell membrane.

TABLE 1

| Tumor associated | | |
|---|---|---|
| antigen | Tumor | Reference |
| MUC-1 | Breast, pancreas, ovarian, | Croce et al. Anticancer |
| | cancer | Res. 17 (1997), |
| | | 4287-92 |
| HER2/neu | Breast, ovarian | Kirpotin at al. |
| (ERBB2)receptor | endometrial, lung, gastric, | Biochem. 36 (1997) |
| | bladder, prostate | 66-75 |
| CEA (carcinoem- | Colon, lung | Jessup et al. Semin. |
| bryonic antigen) | | Surg Oncol 15 (1998) |
| | | 131-140 |
| Folate receptor | Ovarian | Gottschalk et al. Gene |
| | | Therapy 1 (1994) |
| | | 185-91 |
| EGF receptor | Lung | Christiano et al. |
| | | Cancer Gene Ther 3 |
| | | (1996) 4-10 |
| Melanocortin | Melanoma | Szardenings et al. J |
| receptor 1 | | Biol Chem 272 (1997) |
| | | 27943-8 |
| Integrin alpha v | Tumor vessels | Varner et al. Curr |
| beta 3 | | Opin Cell Biol 8 |
| | | (1996) 724-30 |

At the end of any of the above described selection procedures, a limited number of retained phages are individually isolated, amplified and subsequently characterized by determining the sequence of the DNA insert encoding the display peptide. In addition, the amino acid sequences will be aligned to identify motifs that are unique for a given cell. The most abundant sequences are then tested for specific binding.

To confirm the binding specificity of a selected phage in vivo, it will be injected into mice and different organs will be recovered. The accumulation of phages in a given organ will be followed by determining the number of phages recovered from various organs or by performing quantitative PCR for phage specific genomic sequences. The ratio of target/non target organ for the phage or DNA recovery represents a measurement for its specificity. In the literature, ratios of 2 to 35 have been described (Arap et al. Science 279 (1998) 377; Pasqualini et al. Nat. Biotech. 15 (1997) 542; U.S. Pat. No. 5,622,699). This ratio will be compared with the one obtained with unselected phage pools, unselected individual phages or wild type phages. Phage accumulation can also be followed by immunohistochemistry using anti-M13 antibodies. This aspect is particularly relevant to identify more precisely the target tissue (vasculature, tumor cell, ECM). Furthermore, selected phages could be injected in the presence of the free peptide or a GST (gluthation S transferase) fusion peptide to demonstrate specific targeting in a competition assay. In addition, specificity can be tested by linking a tumor-targeting peptide to a chemotherapeutic drug (i.e. doxorubicin) and demonstrating efficiency and selectivity in tumor cell killing.

### EXAMPLE 1

### In vivo Injection of Phase Display Library and Recovery of Organs and Phages

Phage libraries are commercially available. Two of them sold by New England Biolabs were used. PhD-12 contains phages with random 12 amino acid sequences displayed by the pIII protein. PhD-12 stock titer is 1.3×10¹² pfu in 100 μl. Its complexity is 2.7×10⁹. PhD-C7C library displays random 7 mer amino acid sequences flanked by two cysteines displayed by the pill protein. The PhD-C7C stock titer is 1.5×10¹² pfu in 100 82 l with a complexity of 3.7×10⁹. Thus, injection of 5 μl of both libraries should contain at least 20 copies of each phage.

Before being injected into mice, 5 82 l of PhD-12 phages were diluted in 200 μl of DMEM medium (Gibco BRL) (12-D) or in 200 μl of PBS (Dulbecco) (12-P). In parallel, 5 μl of PhD-C7C phages were diluted in 200 μl DMEM (7-D) or in 200 μl PBS (Dulbecco) (7-P).

Mice were anesthesized and the phage dilutions (12-D, 12-P, 7-D, 7-P) were injected through the tail vein (t=o min). Then, mice were perfused through heart with DMEM or PBS (t=2 min). Right after perfusion, and while in deep anesthesia mice were “snap-freezed” in liquid nitrogen.

Analysis is made on organ samples obtained from the injected animals that have been thawed partly at room temperature. Liver, heart, lung, spleen, kidney, and leg muscle were retrieved and placed into 1 ml of ice cold DMEM+PI or PBS+PI (PI is a protease inhibitor cocktail provided by Boehringer ref 1697498). Hearts and livers were lightly grounded with Polytron in an ice/water bath. The tissues were washed 3 to 5 times with 5-10 ml of ice cold DMEM+PI, 1% BSA, 0.1% Tween-20, or PBS+PI, 1% BSA, 0.1% Tween-20. Selected phages were eluted by competition with bacteria. For this purpose, recovered tissues were incubated with 1 ml of early log-phase E.coli ER2537 (New England Biolabs, ref 8110), 20 min at room temperature, with slow shaking. 10 ml of LB medium were added and the whole volume was incubated 20 min at room temperature, with shaking. An aliquote (10 μl) was used for phage titration (Maniatis, Laboratory Manual (1989),Cold Spring Harbor, Laboratory Press) whereas the rest was added to 10 ml of LB medium in an 250 flask. After addition of 150 μl of an overnight culture of ER2537, the culture was incubated 4.5 h with vigorous shaking at 37° C. The culture was centrifuged 10 min at 10 krpm (SS34) at 4° C. two times. 80% of the supernatant was collected and added to ⅙ vol (2.66 ml) of 20% (w/v) PEG-8000, 2.5 M NaCl. Phages were precipitated overnight at 4° C. in order to recover a concentrated stock of the selected phages that was subsequently titered according to the precited technique.

This selection protocol was done three times, before single plaques were picked for DNA isolation and sequencing.

From each round of selection described in example 1, the total number of recovered phages was calculated per 150 mg tissue from each of the organs. The recovered phages were titered before the amplification steps. When phages are recovered from a specific organ in a selection round, it is expected that the recovery from the same organ will increase in the next selection round, but the recovery should not increase from the other organs. FIG. 1 shows the results obtained from an in vivo selection targeting heart in Balb/c mice. Generally, an increase of the phage recovery from the target organ is observed for each selection round. After the three rounds of selection, fifty random phages were picked for sequencing. Table 2 represents a selection of peptides and their frequency of recovery within a selected phage pool. The number indicates the number of times the sequence was found/the number of sequences done in total in the particular experiment.

TABLE 2

| | |
|---|---|
| Peptide sequence | Frequency |

Heart selection

| | |
|---|---|
| THPRFAT | 10/50 |
| HWAPSMYDYVSW | 12/50 |
| QTSSPTPLSHTQ | 5/50 |
| HLPTSSLFDTTH | 4/50 |
| YPSAPPQWLTNT | 4/50 |
| HVNKLHG | 3/50 |
| SGRIPYL | 3/50 |
| LSPQRASQRLYS | 3/50 |
| WKSELPVQRARF | 3/50 |
| HFTFPQQQPPRP | 3/50 |

Tumor selection
B16:

| | |
|---|---|
| TQSPLNYRPALL | 6/50 |
| THRPSLPDSSRA | 5/50 |
| SFPTHIDHHVSP | 4/50 |
| DAQQLYLSNWRS | 3/30 |

P815:

| | |
|---|---|
| MHNVSDSNDSAI | 4/50 |

Liver selection

| | |
|---|---|
| GHLIPLRQPSHQ | 6/30 |

### EXAMPLE 2

### Analysis of Specificity of Candidate Phases in the Heart

Stocks were made of these candidate phages and specificity tested in vivo by IV injection, recovery of target and control organs and calculation of total candidate phages recovered per gram tissue. Alternatively, candidate phages were injected with a negative control phage which yields white plaques instead of blue plaques. The ratio candidate/control recovery is then compared between target and control organs. In the literature, ratios of 2 to 35 have been described (Rajotte et al. J. Clin. Invest. 102 (1998) 430). FIG. 2 shows an example of in vivo testing of specificity of candidate phages with a co-injected negative control. The sequence of the displayed peptide is shown in the figure. In both cases, a higher recovery is found in the target organ (heart) than in the control organ.

### EXAMPLE 3

### Incubation of Fixed Organs with Subtracted Phage Display Library and Recovery of Phases

Subtraction: Phages were preincubated on non target cells, such as Hela (ATCC CCL-2) or 293 (ATCC CRL-1573). For this purpose, cells were grown to confluency in a flask (≧6.3×10⁶ cells) before being fixed in PBS, 0.05% glutaraldehyde for 10 min. The fixed cells were washed 5 times with PBS, 1% BSA to remove glutaraldehyde. 5 μl of the phage display library were diluted in PBS, 1% BSA (2.4 ml, or in smallest volume that covers the plate), added to the fixed cells and incubated 1 h at room temperature with slow rotation. The supernatant containing the subtracted phage suspension was collected by centrifugation 3 min at 1.5 krpm.

Preparation of subtractor cells in suspension: The cells were washed in PBS and detatched with 2 mM EDTA in PBS. After centrifugaion, the cells were resuspended in PBS, 0.05% glutaraldehyde, 1 mM MgCl₂ for 10 min. the cells were washed 5 times with PBS, 1% BSA, 1 mM MgCl₂, and stored in PBS, 1% BSA (6.3×10⁶ cells/ml or higher).

After anesthesia of a Balb/c mouse, organs were mildy fixed by total body perfusion with PBS, 0.05% glutaraldehyde for 10 min. Liver, heart, lung, spleen, kidney, and leg muscle were retieved. Liver and heart were minced with scissors and the fragments were kept at 4° C. in 1 ml PBS, 1% BSA in a polystyrene tube. The other organs were frozen at −80° C. The phage dilution mixed to 6.3×10⁶ subtractor cells was added and incubated overnight at 4° C. with slow rotation.

The supernatant was discarded and the organ fragments were washed with PBS, 1% BSA, 0.05% Tween-20 (5 times). The fragments were kept in 300 μl of wash buffer in a 15 ml tube and the selected phages were eluted at low pH by adding 450 μl of 50 mM Na-citrate, 140 mM NaCl, pH 2.0 for 5 min. Neutralization was made by adding 57 μl of 2 M Tris pH 8.7.

Titration was made on an aliquote (1-10 82 l), and the rest of the supernatant was added to 20 ml of LB medium and 200 μl of an overnight culture of ER2537 before being incubated 4.5 h with vigorous shaking at 37° C. After two centrifugations 10 min at 10 krpm (SS-34) at 4° C., 80% of the supernatant was harvested to which ⅙ vol (2.66 ml) of 20% (w/v) PEG-800, 2.5 M NaCl was added. The mixture was precipitated overnight at 4° C. and the concentrated stock of the selected phages was recovered and titered.

This selection protocol is done three times, before single plaques are picked for isolation of DNA and sequencing.

FIG. 3 shows results obtained from an in vivo selection targeting liver and heart in Balb/c mice. Generally, an increase of the phage recovery from the target organ is observed for each selection round.

### EXAMPLE 4

### Incubation of Target Cells with Subtracted Phage Display Library and Recovery of Phages

Subtraction was done with cells that do not express the target molecules (e.g. MUC-1 polypeptide) as described above. However, the total unsubtracted phage display library may also be used.

The phage suspension was added to the target cells (non-fixed or fixed) and incubated (shortly or overnight) at 40° C. (or other temperature) with slow rotation. The supernatant was discarded and the cells were washed 5 times with PBS, 1% BSA, 0.05% Tween-20. The bound phages were eluted at low pH by adding 450 μl of 50 mM Na-citrate, 140 mM NaCl, pH 2.0 for 5 min. The 57 μl of 2 M Tris pH 8.7 was added to neutralize the phage solution.

Titration was made on an aliquote (1-10 μl), and the rest of the supernatant was added to 20 ml of LB medium and 200 μl of an overnight culture of ER2537. The mixture was incubated 4.5 h with vigorous shaking at 37° C. After two centrifugations 10 min at 10 krpm (SS-34) at 4° C., 80% of the supernatant was harvested, added to ⅙ vol (2.66 ml) of 20% (w/v) PEG-8000, 2.5 M NaCl, and precipitated overnight at 4° C. The selected phages were recovered as a concentrated stock, and titered.

This selection protocol was done three times, before single plaques were picked for isolation of DNA and sequencing.

4.1 Isolation of Peptides Exhibiting Specific Binding to MUC-1 Expressing P815 Tumor Cells.

P815 tumor cell binding phages were isolated by first performing three substractions on P815pAG60 (P815 cells transfected with a Neomycin expression cassette), and subsequently three selection-amplification cycles on P815MUC1 cells (P815 cells (ATCC TIB-64) transfected with MUC1 and Neomycin expression cassettes). P815 are mouse mastocytoma cells available at the ATCC collection (ATCC TIB-64). P815pAG60 cells were grown in DMEM supplemented with 10% fetal calf serum (FCS), 2 mM glutamine, 1 mM sodium pyruvate, 40 μg/ml gentamycin and non-essential amino acids. P815MUC1 cells were grown in the same medium with 1 mg/ml G418 to maintain the expression of the MUC1 gene

For the three subtraction steps, 1×10⁷ P815pAG60 cells were incubated with 1.5×10¹¹ phages from the NEB phage libraries PhD-12 or PhD-C7C (catalog no. 8010 and 8020; NEB, Beverly, USA) for 1 hour at room temperature with slight agitation, in 1 ml of PBS-1% BSA. Cells were then collected by centrifugation at 2500 rpm for 3 minutes. An aliquot of the supernatant was kept for titration, and the rest was incubated again with 1×10⁷ P815pAG60 cells a second and a third time without amplification of the phage pools.

For the three selection cycles, the phage pool from the three successive subtractions were incubated with 5×10⁶ P815MUC1 cells for 4 hours at 4° C. with slight agitation, in 1 ml of PBS-1% BSA. The cells were then washed 5 times with 1 ml of PBS-1% BSA-0.1% Tween 20, and transferred to a new tube during the first wash and before elution. Phages bound to P815MUC1 cells were then eluted with 100 μl of 0.1M glycine-HCl pH2.2 for 10 minutes on ice, cells were pelleted by centrifugation, and the supernatant containing the eluted phage was neutralized with 10 μl of 2M Tris-HCl pH8.

Eluted phages were amplified in 20 ml of LB with 200 μl of an overnight culture of ER2537 bacteria (NEB) for 4.5 h at 37° C. under vigorous shaking. Then bacteria were removed by 2 centrifugation steps at 10000 rpm for 10 minutes, and to 16 ml of supernatant 2.33 ml of 20% PEG 8000, 2.5M NaCl was added for overnight precipitation of the phages at 4° C. The supplier's protocol was then followed to grow and titer a concentrated stock of phages. After the 3rd selection cycle on P815MUC1 cells, the ratio of recovered versus input phages increased by an enrichment factor of up to 500 for the selected pool.

32 single phages were isolated from the third selection pool, amplified and their genomes sequenced to deduce the amino acid sequence of their display peptide. The results are shown in Table 3. From the selection of the PhDC7C phages, two different peptide sequences were enriched and thus represented multiple times. In the selected PhD12 pool, five different phage sequences were identified.

TABLE 3
Sequences and frequencies of isolated candidates.

| | Frequency of recovery |
|---|---|
| Sequence | on P815MUC1 cells |
| CNDIGWVRC | 24/32 |
| CWPYPSHFC | 7/32 |
| MPLPQPSHLPLL | 11/32 |
| LPQRAFWVPPIV | 7/32 |
| WPVRPWMPGPVV | 5/32 |
| WPTSPWLEREPA (WPT1) | 2/32 |
| WPTSPWSSRDWS (WPT2) | 1/32 |

Two other phages were isolated using the technique as described above with the exception that elution was performed with an anti-MUC-1 antibody (12C10 which is a subclone of H23 hybridoma described in Keydar et al., 1989, PNAS, 86, 1362-1366). The sequences of the selected phages are CWPMKSLFC (WPM1) and CWPMKSQFC (WPM2).

4.2 Specific Binding of Selected Phages to P815 Cells

The specificity of the selected phage candidates was then tested by incubating individual phages with P815 cells. Binding of candidates was compared to two negative control phages: empty M13 and GHL, a non-selected phage from the PhD12 library. These studies were performed on P815pAG60, P815MUC1 and non-transformed P815 cells using the phage titration assay or immunostaining by FACS (fluorescence activated cell sorting). The results demonstrate that the described selection scheme allowed the isolation of phages which bind specifically and with high affinity to P815 cells when compared to the negative controls.

Titration Assay

1×10⁷ cells were incubated with 1.5×10¹¹ infectious particles from a selected candidate or control phage. Cells were washed and bound phages were eluted and titered as described above.

The results presented in FIG. 4 demonstrate that all candidates bound with at least 100-fold higher affinity to P815MUC1 and P815pAG60 cells than the controls. The WPY peptide exhibited the best affinity for P815 cells, followed by the NDI phage, and then the 12 amino acid peptide phages. All candidates, except WPY, showed at least 3 times higher binding to P815MUC1 than to P815pAG60 cells.

In addition, the candidate phages were incubated under similar experimental conditions with six other murine and human tumor cell lines: the murine carcinoma cell line RENCA (Murphy et al., 1973, J. Natl. Cancer Inst. 50, 1013-1025), a murine melanoma cell line B16 (ATCC CRL-6322), a human cervix carcinoma cell line HeLa (ATCC CCL-2), a human colorectal cancer cell line WiDr (ATCC CRL-218), and two human breast cancer cell lines MDA-MB-435 (ATCC HTB-129) and MDA-MB-231 (ATCC HTB-26) and their binding analysed by titration studies or FACS assays. All of these cell lines were grown in DMEM supplemented with 10% FCS, 2 mM glutamin and 40 μg/ml gentamycin. Except for WPY which exhibited a specific binding to RENCA cells with up to 10000-fold higher affinity than an M13 control phage, all other candidate phages bound these cell lines with the same affinity as an M13 control phage, indicating that they exhibit high specificity for certain tumor cells types, in particular lymphatic tumors. On the contrary, the WPY phage exhibits a high specificity for at least the two tumoral cell lines RENCA and P815 indicating that it may bind to several different tumor cell types.

FACS Assay

5×10⁵ cells per well were placed in a 96-well plate, 10¹¹ phages were added and incubated for 2 hours at 4° C. under shaking. Cells were washed 4 times with 150 μl of FACS buffer (PBS with 1% BSA, 0.1% human γ-globulin, 5 mM EDTA). 100 μl of an anti-fd bacteriophage antibody (Sigma, St Louis, USA; catalog no: B7786) at 1/500 were added to the wells and incubated for 45 minutes at 4° C. Cells were washed 4 times with FACS buffer and incubated for 45 minutes at 40° C. with a goat anti-rabbit IgG (H+L) antibody coupled to FITC (Biotechnology Associates, Birmingham, USA; catalog no: 4052-02) at 1/200. Cells were washed 4 times with FACS buffer and the fluorescence measured with a FACScan (Becton Dickinson, San Jose, USA). The results were analyzed with the Cellquest software.

The specificity of the above described candidates compared to empty M13 was confirmed by FACS analysis on P815 cells. The WPY, MPL and LPQ phages showed high specific binding to P815MUC1 cells as well as to the original non-transfected P815 cell line. All other clones exhibited binding to P815MUC1 cells, but differed in their binding to non-transfected P815 cells.

4.3. Specific Binding of the WPY and LPQ Synthetic Peptides to P815MUC1 Cells.

Synthetic peptides corresponding to the WPY and LPQ sequences of the previously selected phages (second and fourth sequences of Table 3) were synthetized (Neosystem, strasbourg, France). Increasing amounts of WPY peptide (0.1, 10 and 500 μM) and LPQ peptide (0.1, 10 and 1000 μM) or control peptides GHL and SGR ( a non-selected phage from the PhD-C7C library) were diluted in a total volume of 1 ml of PBS-BSA1% with 5.10⁶ P815MUC1 cells and incubated for 1 hour at 4° C. under slight agitation. 1.10¹⁰ WPY or LPQ phages in 200 μl of PBS-1% BSA were added and incubated with the cells and the peptide for two hours at 4° C. under slight agitation. Cells were then washed and bound phages eluted and titered following the same protocol as for the selections. The WPY and LPQ peptides were able to inhibit in a dose-dependant manner the binding of the corresponding phages, whereas the control peptides did not significatively inhibit WPY and LPQ phage binding, showing that the synthetic peptides efficiently compete for the binding of the phages displaying the same sequence. These resultes indicate that synthetic peptides representing the WPY and LPQ sequences also exhibited specific binding to P815 MUC-1 cells.

Interestingly, the WPY peptide (500 μM concentration) did not significatively inhibit the binding of the LPQ phage, indicating that these two peptides recognize different molecular targets.

### EXAMPLE 5

### Isolation of Phases Exhibiting Specific Binding to WiDr (Human Colorectal Carcinoma Cells)

All cells originated from the ATCC collection and were maintained in DMEM, supplemented with 10% fetal calf serum (FCS), 2 mM glutamine, and 40 μg/ml gentamycin.

The supplied PhD-12 or PhD-C7C library was first preadsorbed on HeLa cells three times before the first selection on WiDr cells. The HeLa cells were brought in suspension by incubating in PBS, 10 mM EDTA. The cells were then washed twice by adding 10 ml PBS, and collected by centrifugation (2500 rpm for 3 min). The cells were counted and resuspended in 1 ml PBS, 1% BSA, per 10⁷ cells.

1.5×10¹¹ pfu from the phage library were added to 1 ml of cells, and the mix incubated for 1 h at room temperature, with slow shaking. After centrifugation at 2500 rpm for 3 min, the supernatant was incubated again with 10⁷ HeLa cells. This subtraction protocol was repeated 3 times. The final supernatant (the subtracted pool of phages) was then incubated with 5×10⁶ WiDr cells in suspension for 4 hours at 4° C., with slow shaking. The cells were washed five times in 1 ml cold PBS, 1% BSA, 0.1% Tween-20, and collected as above. The bound phages were eluted by adding 100 μl 0.1 M Glycine-HCl, pH 2.2, and incubating 10 min on ice. After centrifugation at 2500 rpm for 3 min, the supernatant was neutralized with 10 μl 2 M Tris-HCl, pH. An aliquot of 10 μl was titered and the rest was amplified by adding the eluted phages to 20 ml LB with 200 μl overnight E. coli ER2537 culture. The culture was incubated with strong agitation for 4 h and phage purification was performed according to the providers protocol (NEB).

The selection on WiDr cells was repeated 5 times in total, either with no subtraction before the 2^{nd} to 5^{th} selection, or with 3 subtractions on 293 cells before each selection. Twenty four single phages from the final selected pools were amplified and sequenced to identify the peptide sequence.

TABLE 4
Sequences and frequencies of candidate phages selected from WiDr
cells.

| Subtraction/ | | | |
|---|---|---|---|
| Selection | Cells | Sequence | Frequency |
| 1^{st} subtraction | HeLa | | |
| 1^{st} selection | WiDr | | |
| 2^{nd}-5^{th} subtraction | 293 | HEWSYLAPYPWF | 13 of 24 |
| 2^{nd}-5^{th} selection | WiDr | | |
| 1^{st} subtraction | HeLa | | |
| 1^{st}-5^{th} selection | WiDr | QIDRWFDAVQWL | 24 of 24 |
| 1^{st} subtraction | HeLa | | |
| 1^{st} selection | WiDr | | |
| 2^{nd}-5^{th} subtraction | 293 | CLPSTRWTC | 24 of 24 |
| 2^{nd}-5^{th} selection | WiDr | | |

The specificity of the selected phages was tested by binding to WiDr cells in comparison to the binding of the M13 wild type phage, and in comparison to the binding to different tumor cells lines. The binding was done as described above for the selection.

FIG. 5 shows output/input ratios of the HEWSYLAPYPWF phage when binding was tested on different cells, compared to the M13 wild type phage. The HEW phage shows a 1900-fold higher affinity to WiDr cells than the M13 wild type phage, and a 270-fold higher affinity to MDA-MB-435 cells, while the affinity to 293, and HeLa cells is similar to the M13 wild type affinity.

In a parallel experiment, five selections were made on WiDr cells, but subtraction of the library was only done on HeLa cells before the first selection on the WiDr cells. Selected phages were collected after each of the five rounds of WiDr cell selection (pool 1 to pool 5). From the fifth pool, 24 single plaques were amplified and the insert, corresponding to the peptide, was sequenced. The QIDRWFDAVQWL sequence was obtained from all phages. The purified <<QID>>-phage, and the fifth pool, was found to have affinity to various tumor cell lines, in contrast, the unselected pool 1 did not show affinity to the tumor cell lines. These results demonstrate an affinity of the QID phage to several different tumor cell types.

The same protocol as for selection of the <<HEW>>-phage was repeated with the pHD-C7C library. The fifth pool from this selection contained phages displaying the sequence CLPSTRWTC and showed specific binding to WiDr cells compared to the subtractor 293 cells.

1. A peptide selected from the group consisting of:
X₁CNDIGWVRCX₂
X₁CWPYPSHFCX₂
X₁MPLPQPSHLPLLX₂
X₁LPQRAFWVPPIVX₂
X₁WPVRPWMPGPVVX₂
X₁WPTSPWLEREPAX₂
X₁WPTSPWSSRDWSX₂
X₁HEWSYLAPYPWFX₂
X₁QIDRWFDAVQWLX₂
X₁CLPSTRWTCX₂
X₁CWPMKSX₅FCX2
wherein each X₁
and X₂ independently of one another represents any amino acid sequence of n amino acids, n varying from 0 to 50 and n being identical or different in X₁ and X₂, and wherein X₅ represents any amino acid.

2. Use of a peptide according to claim 1, for targeting a target cell.

3. A tumor targeting peptide comprising at least a three amino acid motif selected from the group consisting of:
(i) NDI, WPY, MPL, PSH, LPQ, WPV or WPT or any combination thereof, and
(ii) HEW, QID, WPM or CLP or any combination thereof.

4. A peptide according to claim 3, having the following sequence:
(i) X₁CNDIGWVRCX₂,
X₁CWPYPSHFCX₂,
X₁MPLPQPSHLPLLX₂,
X₁LPQRAFWVPPIVX₂ ,
X₁WPVRPWMPGPVVX₂,
X₁WPTSPWLEREPAX₂,
X₁WPTSPWSSRDWSX₂, or
(ii) X₁HEWSYLAPYPWFX₂
X₁QIDRWFDAVQWLX₂
X₁CLPSTRWTCX₂
X₁CWPMKSX₅FCX2
wherein each X₁ and X₂ independently of one another represents any amino acid sequence of n amino acids, n varying from 0 to 50 and n being identical or different in X₁ and X₂ and wherein X₅ represents any amino acid.

5. Use of a peptide according to claim 3 or 4, for targeting to a tumor cell, a metastasis or a tumor vasculature.

6. Use of a peptide according to claim 4 selected among the group consisting of X₁HEWSYLAPYPWFX₂, X₁QIDRWFDAVQWLX₂ and X₁CLPSTRWTCX₂, for targeting to a colorectal tumor cell.

7. Use of a peptide X₁CWPYPSHFCX₂ according to claim 4, for targeting to a carcinoma tumor cell.

8. A composition comprising at least one peptide according to any one of claims 1 to 4 and at least one therapeutic agent or alternatively at least one nucleic acid molecule encoding a peptide according to any one of claims 1 to 4 and at least one therapeutic agent.

9. The composition according to claim 8, wherein said therapeutic agent is a vector for delivering at least one gene of interest to a target cell of a vertebrate.

10. The composition according to claim 9, wherein said vector is a plasmid, a synthetic or a viral vector.

11. The composition according to any of claims 8 to 10, wherein said peptide is operably coupled to said therapeutic agent by covalent, non covalent or genetic means.

12. Use of a composition according to any of claims 8 to 11, for the preparation of a drug intended for gene transfer.
